(19) United States
(12) Patent Application Publication
(10) Pub. No.: US 2016/0067172 A1
(43) Pub. Date: Mar. 10, 2016

(54) Sulfate-Free Personal Care Compositions and Methods

(71) Applicant: Elementis Specialties, Inc., East Windsor, NJ (US)

(72) Inventors: Kimberly Burch, Mercerville, NJ (US); Binhua Yang, Pennington, NJ (US); Timothy Kenny, Ringoes, NJ (US)

(73) Assignee: Elementis Specialties, Inc., East Windsor, NJ (US)

(21) Appl. No.: 14/845,897

(22) Filed: Sep. 4, 2015

Related U.S. Application Data
(60) Provisional application No. 62/047,360, filed on Sep. 8, 2014.
(60) Provisional application No. 62/047,924, filed on Sep. 9, 2014.

(51) Int. Cl. A61K 8/87; A61Q 19/10; A61K 8/20; A61Q 5/02; A61K 8/44; A61K 8/46

(52) U.S. Cl. CPC A61K 8/87; A61K 8/442; A61K 8/466; A61K 8/20; A61Q 5/02; A61Q 19/10; A61K 2800/48

(57) ABSTRACT

A personal care composition includes a surfactant; and a polymer comprising a hydrophobically modified polyurethane, a hydrophobically modified polyether polyol, or a combination thereof; wherein the personal care composition has a viscosity ranging from about 8,000 cP to about 40,000 cP; and wherein the personal care composition is free of sulfates

### CROSS-REFERENCE TO RELATED APPLICATIONS

This application claims the benefit of U.S. Provisional Patent Application No. 62/047,360 filed 8 Sep. 2014 entitled “Sulfate Free Hair Care Composition,” and U.S. Provisional Patent Application No. 62/047,924 filed 9 Sep. 2014 entitled “Sulfate Free Hair Care Composition,” which are each incorporated by reference herein in their entirety.

### BACKGROUND OF THE INVENTION

Historically, shampoos typically utilize anionic surfactants, such as sulfate-based surfactants, and so historically the viscosity of the shampoo has been easy to control, for example by addition of salt. However, sulfate based surfactants have a tendency to dry out hair, strip color-treated hair of dye, and break down proteins such as keratin. Sulfate-free surfactants are becoming increasingly desirable to consumers, but traditional methods of increasing viscosity, such as incorporation of a salt, generally have no effect on the viscosity of sulfate free personal care solutions.

The present invention addresses these shortcomings.

### BRIEF SUMMARY OF THE INVENTION

In one embodiment the present invention provides personal care composition comprising a surfactant; and a polymer comprising a hydrophobically modified polyurethane, a hydrophobically modified polyether polyol, or a combination thereof; wherein the personal care composition has a viscosity ranging from about 8000 cP to about 40,000 cP; and wherein the personal care composition is free of sulfates. The surfactant can be non-ionic, anionic, cationic, amphoteric, or zwitterionic. The personal care composition may include more than one different type of surfactant, which may each independently be selected from non-ionic, anionic, cationic, amphoteric, or zwitterionic surfactants. In some embodiments the surfactant and the polymer are present in a weight ratio of about 0.25:1 to about 3:1. In some embodiments, the surfactant is present in an amount ranging from about 0.1% to about 40% by weight of the personal care composition. In some embodiments the polymer is present in an amount ranging from about 1% to about 4% by weight of the personal care composition.

In some embodiments the hydrophobically modified polyurethane is selected from condensates of: (i) at least one polyoxyalkylene with a number average molecular weight ranging from 2,000-10,000 g/mole; (ii) a linear, branched, or alicylic aliphatic alcohol having an aliphatic group with 6 to 18 carbon atoms, said aliphatic group optionally linked to a polyoxyalkylene group with a number average molecular weight ranging from 2,000-10,000 g/mole; and (iii) an aliphatic diisocyanate compound. The condensate may further comprise a diol having 6 to 16 carbon atoms. In some embodiments, the linear or branched aliphatic alcohol may be independently selected from the group consisting of: 1-hexanol, 2-ethylhexanol, 1-octanol, 1-decanol, 1-dodecanol, 1-tetradecanol, 1-hexadecanol, 1-octadecanol, 2-butyloctanol, 2-hexyldecanol, 2-octyldecanol, 1-isononanol, 1-isooctadecanol, 1-isoundecanol, 1-isotridecanol and mixtures thereof. In some embodiments, the diisocyanate compound may be independently selected from the group consisting of: hexamethylene diisocyanate, trimethyl hexamethylene diisocyanate, isophorone diisocyanate, tetramethyl xylylene diisocyanate, 4,4′-methylene bis(cyclohexylisocyanate) and mixtures thereof.

In some embodiments the hydrophobically modified polyurethane is a condensate of a polyethylene glycol with a number average molecular weight of about 4500-6500 g/mole, a linear aliphatic alcohol having an aliphatic group with 18 carbon atoms, said aliphatic group linked to a polyoxyalkylene group with a number average molecular weight of about 3000-5000 g/mol; and hexamethylene diisocyanate.

In some embodiments the hydrophobically modified polyether polyol is selected from condensates of: (i) an acyclic polyhydric alcohol having 4 to 6 carbon atoms and 4 to 6 reactive hydroxyl groups; a cyclic polyhydric alcohol having 4 to 6 carbon atoms and 4 to 6 reactive hydroxyl groups, and combinations thereof; (ii) polyoxyalkylene with a number average molecular weight ranging from 2,000-10,000 g/mole; and (iii) a linear, branched, or alicylic aliphatic epoxide having an aliphatic group with 6 to 18 carbon atoms. In some embodiments, the acyclic polyhydric alcohol may be selected from pentaerythritol, erythritol, and combinations thereof. In some embodiments, the polyoxyalkylene has alternating groups selected from (OCH₂CH₂); —(OCH(CH₃)CH₂, —(OCH₂(CH₂)CH₃ and combinations thereof.

In a further embodiment, the present invention provides a method of increasing the viscosity according to a personal care composition described herein. Such a method includes the steps of providing a personal care composition comprising at least one surfactant and a polymer comprising a hydrophobically modified polyurethane, a hydrophobically modified polyether polyol, or a combination thereof; and adding a salt to the personal care composition to increase the viscosity of the personal care composition. In some embodiments, the salt may be NaCl. The amount of salt added to the composition may be sufficient to provide the composition with a viscosity between about 8000 cP and about 40000 cP.

In yet another embodiment, the present invention provides a method of making a personal care composition described herein. Such a method includes the steps of (i) mixing a polymer comprising a hydrophobically modified polyurethane, a hydrophobically modified polyether polyol, or a combination thereof in water to form a phase A; heating the phase A to between about 60° C. and about 100° C.; mixing at least one surfactant into phase A to form a personal care composition; and cooling the personal care composition. In an embodiment the method further comprises the step of mixing an amount of salt to the personal care composition after the cooling step to increase the viscosity of the personal care composition. In some embodiments, the salt may be NaCl. In some embodiments the amount of salt added to the composition may be sufficient to provide the composition with a viscosity between about 8000 cP and about 40000 cP.

### DETAILED DESCRIPTION OF THE INVENTION

The present disclosure provides for sulfate-free personal care compositions and a method of making sulfate-free personal care compositions which result in a viscosity that is desirable to consumers. It has surprisingly been found that personal care compositions of the present invention may have a viscosity that is desirable to consumers while being free of sulfates, regardless of the type of surfactant included in the composition. Advantages of the sulfate-free personal care compositions of the present invention may include an outstanding sensory profile, for example, a smooth formulation that is soft to the touch with no tacky or negative aesthetics. The sulfate-free personal care compositions of the present invention may be compatible with anionic, non-ionic, cationic, amphoteric, zwitterionic emulsifiers, or combinations thereof. Moreover, the sulfate-free personal care compositions of the present invention may be compatible in systems with extreme pH or high electrolyte concentration. The sulfate-free personal care compositions of the present invention do not have a negative impact on foam stability, another characteristic of personal care compositions of significance to consumers.

A sulfate free personal care composition of the present invention may comprise a surfactant and a hydrophobically modified polymer, wherein the personal care composition is free of sulfates. “Sulfates,” as the term is used herein, is limited to compounds that include a sulfate moiety (SO₄⁻²); a composition may include sulfate derivatives, such as sulfonates, and still be considered “sulfate free” or “free of sulfates” as those terms are used herein.

Personal care compositions of the present invention comprise a surfactant. Any surfactant that is free of sulfates can be used in the present invention. Suitable surfactants include non-ionic, anionic, cationic, amphoteric, and zwitterionic surfactants, or combinations thereof. In some embodiments a personal care composition comprises more than one surfactant. Combinations of multiple sulfate-free surfactants are within the scope of the invention. In such embodiments, each surfactant may be independently chosen from non-ionic, anionic, cationic, amphoteric, and zwitterionic surfactants.

The various embodiments of personal care compositions described herein may have a viscosity ranging from about 4,000 cP to about 60,000 cP, from about 8000 cP to about 40,000 cP; or about 10,000 cP to about 20,000 cP. In some embodiments the viscosity of a personal care composition described herein is greater than about 8,000 cP, greater than about 10,000 cP, greater than about 15,000 cP, greater than about 20,000 cP, greater than about 25,000 cP, greater than about 30,000 cP, greater than about 35,000 cP, greater than about 40,000 cP, greater than about 45,000 cP, greater than about 50,000 cP, greater than about 55,000 cP, greater than about 60,000 cP, greater than about 70,000 cP, greater than about 80,000 cP, greater than about 90,000 cP, or greater than about 100,000 cP.

Suitable anionic surfactants for use in the present invention include alkylsulfonates, alkylamidesulfonates, alkylarylsulfonates, α-olefin sulfonates, paraffin sulfonates; alkyl sulfosuccinates, alkyl ether sulfosuccinates, alkylamide sulfosuccinates; alkyl sulfoacetates; acylsarcosinates; and acylglutamates, or salts, in particular the alkali metal salts such as the sodium salts, the ammonium salts, the amine salts, the amino alcohol salts or the salts of alkaline-earth metals, for example of magnesium, thereof. The alkyl and acyl groups of such anionic surfactants may comprise from 6 to 24 carbon atoms and the aryl group may preferably denote a phenyl or benzyl group. Suitable anionic surfactants for use in the present invention further include acyl lactylates, the acyl group of which comprises from 8 to 20 carbon atoms. In addition, suitable anionic surfactants may also be made of alkyl-D-galactosideuronic acids and salts thereof and also polyoxyalkylenated (C₆₋₂₄)alkyl ether carboxylic acids, polyoxyalkylenated (C₆₋₂₄)alkyl(C₆₋₂₄)aryl ether carboxylic acids, polyoxyalkylenated (C₆₋₂₄)alkylamido ether carboxylic acids and salts thereof, in particular those comprising from 2 to 50 ethylene oxide units, and mixtures thereof.

Suitable nonionic surfactants for use in the present invention include polyethoxylated, polypropoxylated or polyglycerolated alcohols, alpha-diols, (C₁₋₂₀)alkylphenols and fatty acids, containing a fatty chain comprising, for example, from 8 to 18 carbon atoms, the number of ethylene oxide or propylene oxide groups possibly ranging especially from 2 to 50, and the number of glycerol groups possibly ranging especially from 2 to 30. Suitable nonionic surfactants for use in the present invention further include condensates of ethylene oxide and of propylene oxide with fatty alcohols; polyethoxylated fatty amides preferably having from 2 to 30 ethylene oxide units, polyglycerolated fatty amides containing on average 1 to 5 glycerol groups, and in particular 1.5 to 4, ethoxylated fatty acid esters of sorbitan containing from 2 to 30 ethylene oxide units, fatty acid esters of sucrose, fatty acid esters of polyethylene glycol, (C₆₋₂₄alkyl)polyglycosides, (C₈₋₁₈ alkyl)polyglycosides, N—(C₆₋₂₄ alkyl)glucamine derivatives, amine oxides such as (C₁₀₋₁₄alkyl)amine oxides or N—(C₁₀₋₁₄ acyl)aminopropylmorpholine oxides.

Suitable amphoteric surfactants for use in the present invention include secondary or tertiary aliphatic amine derivatives in which the aliphatic group is a linear or branched chain containing from 8 to 22 carbon atoms and containing at least one anionic group, for instance a carboxylate, sulfonate, sulfate, phosphate or phosphonate group. Suitable amphoteric surfactants for use in the present invention further include (C₈₋₂₀)alkylbetaines, sulfobetaines, (C₈₋₂₀ alkyl)amido(C₂₋₈ alkyl)betaines and (C₈₋₂₀ alkyl)amido(C₂₋₈ alkyl)sulfobetaines. Suitable amphoteric surfactants for use in the present invention further include alkylamphodiacetates, amphocarboxyglycinate and amphocarboxypropionate, disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, cocoamphodipropionic acid.

Preferably, a surfactant for use in the present invention can include one or more of the following: sodium C₁₄₋₁₆ alpha sulfonate (alpha olefin sulfonate), sodium C₁₄₋₁₇ alkyl sulfonate (alkyl sulfonate), coco-glucoside polyalkyl glycoside C₈₋₁₆ (Surfapon AG-818), disodium laureth sulfosuccinate (sulfosuccinate), cocoglucoside (Plantacare® 818), Decyl polyglucoside (polyglucoside), meadowfoamamidopropyl betaine (Betafan-M), cocoamidopropyl hydroxysultaline (Surfatex CBS), sodium cocoyl isethionate (SCI 85% Noodle). cocoamidopropyl betaine (Tego® betain F50), decyl glucoside (Plantacare® 1000UP).

In some embodiments the surfactant is present in an amount ranging from about 0.1% to about 80%, about 0.1% to about 70%, about 0.1% to about 60%, about 0.1% to about 50%, about 0.1% to about 40%, about 1% to about 60%, about 10% to about 50%, or about 20% to about 40% by weight of the personal care composition. In some embodiments the surfactant is present in an amount of about 10%, about 15%, about 20%, about 25, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 55%, or about 60% by weight of the personal care composition.

A hydrophobically modified polymer that is suitable for use in the compositions and methods of the present invention may function as an associative thickener. That is, suitable polymers may increase the viscosity of an aqueous system through a system of molecular associations and interactions. In an embodiment an associative thickener comprises two or more terminal hydrophobic regions with a hydrophilic chain extending between the terminal hydrophobic regions. Without being bound by theory, the terminal hydrophobic regions of the polymer associate, thereby building a structured network. In some embodiments other the structured network further comprises other hydrophobes that may be present in the formula. Association of the terminal hydrophobic regions, and hydrophobes if present, may result in one or more micelle type structures that may form a network, resulting in a significant viscosity build.

In one embodiment, the polymer is a hydrophobically modified polymer. In some embodiments the hydrophobically modified polymer may comprise a hydrophobically modified polyurethane, a hydrophobically modified polyether polyol, or a combination of hydrophobically modified polyurethane and hydrophobically modified polyether polyol. In some embodiments the hydrophobically modified polymer may comprise polyurethane linkages, or polyether polyol linkages. In one such embodiment, the hydrophobically modified polymeric chain is derived from a polyethylene oxide polymer, a polypropylene oxide polymer, or a polyethylene oxide-polypropylene oxide copolymer, hereinafter designated polyoxyalkylene chains or polyoxyalkylene polymers.

A hydrophobically modified polyurethane polymer may be selected from condensates of (i) at least one polyoxyalkylene (ii) a linear, branched, or alicylic aliphatic alcohol having an aliphatic group, said aliphatic group optionally linked to a polyoxyalkylene group; and (iii) an aliphatic diisocyanate compound. For example, in an embodiment, a hydrophobically modified polyurethane may be selected from condensates of (i) at least one polyoxyalkylene with a number average molecular weight ranging from 2,000-10,000 g/mole; (ii) a linear, branched, or alicyclic aliphatic alcohol having an aliphatic group with 6 to 18 carbon atoms, said aliphatic group optionally linked to a polyoxyalkylene group with a number average molecular weight ranging from 2,000-10,000 g/mole; and (iii) an aliphatic diisocyanate compound.

In some embodiments the hydrophobically modified polyurethane is a condensate of a polyethylene glycol with a number average molecular weight of about 4500-6500 g/mole, a linear aliphatic alcohol having an aliphatic group with 18 carbon atoms, said aliphatic group linked to a polyoxyalkylene group with a number average molecular weight of about 3000-5000 g/mole; and hexamethylene diisocyanate. In some embodiments the hydrophobically modified polyurethane is a condensate of a polyethylene glycol with a number average molecular weight of about 3,000-8,000 g/mole, 4,000-7,000 g/mole, or about 4500-6500 g/mole, a linear aliphatic alcohol having an aliphatic group with 15, 16, 17, 18, 19, or 20 carbon atoms, said aliphatic group linked to a polyoxyalkylene group with a number average molecular weight of about 2,000-8,000 g/mole, about 2,000-6,000 g/mole, about 2500-4500 g/mole; about 3,000-7,000 g/mole, about 3,000-5,000 g/mole, about 4,000-7000 g/mole or about 4,500-6,500 g/mole and hexamethylene diisocyanate.

“Oxyalkylene” refers to units having the structure —(O-A)-, with O-A represents the monomeric residue of the polymerization reaction product of C₂₋₈ alkylene oxides or diols. Examples of oxyalkylenes include, but are not limited to: oxyethylene with the structure (OCH₂CH₂); oxypropylene with the structure —(OCH(CH₃)CH₂— or —(OCH₂(CH)(CH₃)—; oxytrimethylene with the structure —(OCH₂CH₂CH₂)—; and 1,4-oxybutylene with the general structure —(OC₄H₈)—.

“Polyoxyalkylene” refers to a polymer containing oxyalkylene units. The polyoxyalkylene units can be homopolymeric or copolymeric. Examples of homopolymers of polyoxyalkylenes include, but are not limited to polyoxyethylene, which contains units of oxyethylene; polyoxypropylene, which contains units of oxypropylene; polyoxytrimethylene, which contains units of oxytrimethylene; and polyoxybutylene, which contains units of oxybutylene. Examples of polyoxybutylene include a homopolymer containing units of 1,2-oxybutylene, —(OCH(C₂H₅)CH₂)—; and polytetrahydrofuran, a homopolymer containing units of 1,4-oxybutylene, or —(OCH₂CH₂CH₂CH₂). The polyoxyalkylene compounds, independent of monomer composition, may be water soluble.

In other embodiments, the polyoxyalkylene units can be copolymeric, containing two or more different oxyalkylene segments. The different oxyalkylene groups can be arranged randomly to form a random polyoxyalkylene; or can be arranged in blocks to form a block polyoxyalkylene. Block polyoxyalkylene polymers have two or more neighboring polymer blocks, wherein each of the neighboring polymer blocks contain different oxyalkylene segments, and each polymer block contains at least two of the same oxyalkylene segments. In one such embodiment, an oxyalkylene group is oxyethylene.

In some embodiments, the polyoxyalkylene chains have a nominal number average molecular weight ranging from 2,000-10,000 g/mole. The polyoxyalkylene group may have a number average molecular weight ranging from 2,000-8,000 g/mole, from 3,000-6,000 g/mole, or from 4,000-5,000 g/mole. In another embodiment, the polyoxyalkylene chains have a number average molecular weight of up to about 5,000-10,000 g/mole. For example, the polyoxyalkylene group may have a number average molecular weight up to about 6,000 g/mole, 8,000 g/mole, or 9,000 g/mole.

In one embodiment, the linear or branched aliphatic alcohol is selected from the group consisting of 1-hexanol, 2-ethylhexanol, 1-octanol, 1-decanol, 1-dodecanol, 1-tetradecanol, 1-hexadecanol, 1-octadecanol, 2-butyloctanol, 2-hexyldecanol, 2-octyldecanol, 1-isononanol, 1-isooctadecanol, 1-isoundecanol, 1-isotridecanol and mixtures thereof. In another embodiment the aliphatic alcohol is selected from the group consisting of di-n-octylaminoethanol, di-2-ethylhexylaminoethanol, di-hexylaminoethanol, 2-(diphenylphosphino)ethylamine, 2-(dioctylamino)-ethanol, and 2-(diethylhexylamino)-ethanol.

The linear, branched, or alicyclic aliphatic alcohol may have a polyoxyalkylene group linked to the aliphatic group. In some embodiments the polyoxyalkylene group linked to the aliphatic group of a linear, branched, or alicyclic aliphatic alcohol may have a number average molecular weight ranging from about 2,000-10,000 g/mole, from about 2,000-8,000 g/mole, from about 3,000-6,000 g/mole, or from about 4,000-5,000 g/mole.

A variety of aliphatic diisocyante compounds may be used to generate polyurethane linkages of a hydrophobically modified polyurethane. In one embodiment, aliphatic diisocyanate compounds include hexamethylene diisocyanate (“HDI”), trimethyl hexamethylene diisocyanate (“TMDI”), isophorone diisocyanate (“IPDI”), tetramethyl xylylene diisocyanate (“TMXDI”), 4,4′-methylene bis(cyclohexylisocyanate) (“DESDOMUR® W) and mixtures thereof. In some embodiments, the aliphatic diisocyanate compound may be isophorone diisocyanate. In other embodiments, the aliphatic diisocyanate compound may be hexamethylene diisocyanate. In still yet other embodiments, the aliphatic diisocyanate compound may be 4,4′-methylene bis(cyclohexylisocyanate).

A hydrophobically modified polyether polyol may be selected from condensates of: (i) an acyclic polyhydric alcohol having 4 to 6 carbon atoms and 4 to 6 reactive hydroxyl groups; a cyclic polyhydric alcohol having 4 to 6 carbon atoms and 4 to 6 reactive hydroxyl groups, and combinations thereof; (ii) polyoxyalkylene with a number average molecular weight ranging from 2,000-10,000 g/mole; and (iii) a linear, branched, or alicylic aliphatic epoxide having an aliphatic group with 6 to 18 carbon atoms.

A polyoxyalkylene may be any polyoxyalkylene as described herein for a hydrophobically modified polyurethane.

A polyhydric alcohol may be an acyclic polyhydric alcohol, a cyclic polyhydric alcohol, or combinations thereof. In one embodiment, the polyhydric alcohol may be an acyclic polyhydric alcohol having 4 to 6 carbon atoms and 4 to 6 reactive hydroxyl groups, a cyclic polyhydric alcohol having 4 to 6 carbon atoms and 4 to 6 reactive hydroxyl groups, and combinations thereof. In one embodiment the acyclic polyhydric alcohol is selected from the group consisting of pentaerythritol, erythritol, and combinations thereof.

In some embodiments, personal care compositions according to the present invention include surfactant and a hydrophobically modified polymer (e.g., a hydrophobically modified polyurethane, a hydrophobically modified polyether polyol, or a combination of hydrophobically modified polyurethanes and hydrophobically modified polyether polyols) in a weight ratio in a range of about 0.25:1 to about 3:1, or in a range of about 0.1:1 to about 5:1. In some embodiments, personal care compositions according to the present invention include surfactant and a hydrophobically modified polymer (e.g., a hydrophobically modified polyurethane, a hydrophobically modified polyether polyol, or a combination of hydrophobically modified polyurethanes and hydrophobically modified polyether polyols) in a weight ratio of about 0.1:1, about 0.2:1, about 0.25:1, about 0.5:1, about 0.75:1, about 1:1, about 1.5:1, about 2:1, about 2.5:1, about 3:1, about 3.5:1, about 4:1, or about 5:1.

The composition according to the invention may include additional ingredients, such as nacreous agents, opacifiers, dyes or pigments, fragrances, mineral, plant or synthetic oils, waxes, vitamins, UV-screening agents, free-radical scavengers, antidandruff agents, preserving agents, pH stabilizers and solvents, and mixtures thereof.

Brookfield viscosities of such compositions are measured at 25° C. and 10 RPM using a Brookfield RV or RVT. Typically a #1 spindle is used for materials having a Brookfield viscosity of 400-600 cP (40-60 dial reading), a #2 spindle is used for materials having a Brookfield viscosity of 1600-2400 cP and a #3 spindle is used for materials having a Brookfield viscosity of 4000-6000 cP. The following table may also be used to determine the appropriate spindle to use when measuring Brookfield viscosity at 25° C. and 10 RPM:

Viscosity (cP)

| Spindle | Factor | Mid-dial | Max |
|---|---|---|---|
| 1 | 10 | 500 | 1000 |
| 2 | 40 | 2000 | 4000 |
| 3 | 100 | 5000 | 10000 |
| 4 | 200 | 10000 | 20000 |
| 5 | 400 | 20000 | 40000 |
| 6 | 1000 | 50000 | 100000 |
| 7 | 4000 | 200000 | 400000 |

It has been surprisingly found that salts interact synergistically with the hydrophobically modified polymer of the personal care compositions of the present invention to increase the viscosity of the personal care compositions of the invention. This synergistic increase in viscosity allows for control over the viscosity of a personal care composition including any type of surfactant, for example sulfate free surfactants. In some embodiments of the present invention, a personal care composition may further comprise a salt, for example NaCl. Other salts may include ammonium chloride. Salt may be present in 0.1% to about 5%, about 0.1% to about 4%, about 0.1% to about 3%, about 0.5% to about 3%, about 1% to about 3% by weight of the personal care composition. Salt may be present in about 0.1%, about 0.5%, about 1%, about 1.25%, about 1.5%, about 1.75%, about 2%, about 2.25%, about 2.5%, about 2.75%, about 3%, about 3.5%, about 4%, about 4.5%, or about 5% by weight of the personal care composition.

The present invention also provides for method of increasing the viscosity of a personal care composition as described herein. In some embodiments a method of increasing the viscosity of a personal care composition comprises providing a personal care composition as described herein, comprising at least one surfactant and a hydrophobically modified polymer; and adding a salt to the personal care composition to increase the viscosity of the personal care composition. The salt may be added at room temperature after the other ingredients in the personal care composition have been mixed, allowing the viscosity to be increased to a desired level after the personal care composition has been prepared and providing the manufacturer with greater control over the final viscosity of the personal care composition than has traditionally been available with sulfate-free personal care compositions. In some embodiments the salt is added to the composition in an amount sufficient to provide the composition with a viscosity of between about 4,000 cP to about 60,000 cP, from about 8000 cP to about 40,000 cP; or about 10,000 cP to about 20,000 cP. In some embodiments the amount of salt added is sufficient to provide the composition with a viscosity greater than about 8,000 cP, greater than about 10,000 cP, greater than about 15,000 cP, greater than about 20,000 cP, greater than about 25,000 cP, greater than about 30,000 cP, greater than about 35,000 cP, greater than about 40,000 cP, greater than about 45,000 cP, greater than about 50,000 cP, greater than about 55,000 cP, greater than about 60,000 cP, greater than about 70,000 cP, greater than about 80,000 cP, greater than about 90,000 cP, or greater than about 100,000 cP.

The various embodiments of personal care compositions described herein may be made by mixing a polymer comprising a hydrophobically modified polyurethane, a hydrophobically modified polyether polyol, or a combination thereof, in water to form a phase A; heating the phase A to between about 60° C. and about 100° C.; mixing at least one surfactant into phase A to form a personal care composition; and cooling the personal care composition. In some embodiments the method further includes the step of adding a salt to the personal care composition. The salt may be added to the personal care composition after the personal care composition has been cooled, for example to about room temperature. In some embodiments the amount of salt added is sufficient to provide the composition with a viscosity of between about 4,000 cP to about 60,000 cP, from about 8000 cP to about 40,000 cP; or about 10,000 cP to about 20,000 cP. In some embodiments the amount of salt added is sufficient to provide the composition with a viscosity greater than about 8,000 cP, greater than about 10,000 cP, greater than about 15,000 cP, greater than about 20,000 cP, greater than about 25,000 cP, greater than about 30,000 cP, greater than about 35,000 cP, greater than about 40,000 cP, greater than about 45,000 cP, greater than about 50,000 cP, greater than about 55,000 cP, greater than about 60,000 cP, greater than about 70,000 cP, greater than about 80,000 cP, greater than about 90,000 cP, or greater than about 100,000 cP.

### Examples

The following examples further describe and demonstrate illustrative embodiments within the scope of the present invention. The examples are given solely for illustration and are not to be construed as limitations of this invention as many variations are possible without departing from the spirit and scope thereof.

### Example 1

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 1

| | | Ratio | |
|---|---|---|---|
| Betafan-M | | surfactant:hydro- | Viscosity |
| (25% solid) | Polymer #1 | phobically | (cP) RV#6, |
| weight % of | weight % of | modified | 10-50 rpm, |
| composition | composition | polyurethane | 21° C. |
| 1 | 3 | 0.33 | 66900 |
| 2 | 3 | 0.67 | 123750 |
| 3 | 3 | 1.00 | 163400 |
| 4 | 3 | 1.33 | 160333 |
| 5 | 3 | 1.67 | 127700 |
| 1 | 2 | 0.50 | 16800 |
| 2 | 2 | 1.00 | 31166 |
| 3 | 2 | 1.50 | 40900 |
| 4 | 2 | 2.00 | 19000 |
| 5 | 2 | 2.50 | 14300 |

### Example 2

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 2

| Surfapon | | | Viscosity |
|---|---|---|---|
| AG-818 (50%) | Polymer #1 | Ratio | (cP) RV#6, |
| weight % of | weight % of | surfactant:hydrophobically | 10-50 rpm, |
| composition | composition | modified polyurethane | 21° C. |
| 1 | 3 | 0.33 | 28400 |
| 2 | 3 | 0.67 | 54500 |
| 3 | 3 | 1.00 | 38400 |
| 4 | 3 | 1.33 | 17750 |
| 5 | 3 | 1.67 | 11950 |
| 1 | 2 | 0.50 | 3450 |
| 2 | 2 | 1.00 | 10250 |
| 3 | 2 | 1.50 | 7200 |
| 4 | 2 | 2.00 | 4700 |
| 5 | 2 | 2.50 | 3400 |

### Example 3

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 3

| | | Ratio | |
|---|---|---|---|
| Decyl | | surfactant:hydro- | Viscosity |
| polyglucoside | Polymer #1 | phobically | (cP) RV#6, |
| (50%) weight % of | weight % of | modified | 10-50 rpm, |
| composition | composition | polyurethane | 21° C. |
| 1 | 3 | 0.33 | 31200 |
| 2 | 3 | 0.67 | 33800 |
| 3 | 3 | 1.00 | 5200 |
| 4 | 3 | 1.33 | 2300 |
| 5 | 3 | 1.67 | 1100 |
| 1 | 2 | 0.50 | 1300 |
| 2 | 2 | 1.00 | 1900 |
| 3 | 2 | 1.50 | 400 |
| 4 | 2 | 2.00 | 10 |
| 5 | 2 | 2.50 | 100 |

### Example 4

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 4

| Sulfosuccinate | | | Viscosity |
|---|---|---|---|
| (disodium | | | (cP) |
| laureth | | | RV#6, |
| sulfosuccinate) | Polymer #1 | Ratio | 10-50 |
| (32%) weight % | weight % of | surfactant:hydrophobically | rpm, |
| of composition | composition | modified polyurethane | 21° C. |
| 1 | 3 | 0.33 | 22500 |
| 2 | 3 | 0.67 | 20800 |
| 3 | 3 | 1.00 | 11550 |
| 4 | 3 | 1.33 | 3150 |
| 5 | 3 | 1.67 | 500 |
| 1 | 2 | 0.50 | 1550 |
| 2 | 2 | 1.00 | 250 |
| 3 | 2 | 1.50 | 10 |
| 4 | 2 | 2.00 | 10 |
| 5 | 2 | 2.50 | 10 |

### Example 5

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 5

| Alpha olefin | | | Viscosity |
|---|---|---|---|
| sulfonate (sodium | | | (cP) |
| C14-16 alpha | | | RV#6, |
| sulfonate) (40%) | Polymer #1 | Ratio | 10-50 |
| weight % of | weight % of | surfactant:hydrophobically | rpm, |
| composition | composition | modified polyurethane | 21° C. |
| 1 | 3 | 0.33 | 10300 |
| 2 | 3 | 0.67 | 40 |
| 3 | 3 | 1.00 | 20 |
| 4 | 3 | 1.33 | 20 |
| 5 | 3 | 1.67 | 10 |
| 1 | 2 | 0.50 | 10 |
| 2 | 2 | 1.00 | 10 |
| 3 | 2 | 1.50 | 10 |
| 4 | 2 | 2.00 | 10 |
| 5 | 2 | 2.50 | 10 |

### Example 6

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 6

| Alkyl sulfonate | | | |
|---|---|---|---|
| (sodium | | | |
| C14-17 alkyl | Viscosity | | |
| sulfonate) 30% | Polymer #1 | Ratio | (cP) RV#6, |
| weight % of | weight % of | surfactant:hydrophobically | 10-50 rpm, |
| composition | composition | modified polyurethane | 21° C. |
| 1 | 3 | 0.33 | 9400 |
| 2 | 3 | 0.67 | 50 |
| 3 | 3 | 1.00 | 10 |
| 4 | 3 | 1.33 | 10 |
| 5 | 3 | 1.67 | 10 |
| 1 | 2 | 0.50 | 10 |
| 2 | 2 | 1.00 | 10 |
| 3 | 2 | 1.50 | 10 |
| 4 | 2 | 2.00 | 10 |
| 5 | 2 | 2.50 | 10 |

### Example 7

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 7

| Surfatex CBS | | | |
|---|---|---|---|
| (cocaamideopropyl | | | |
| hydroxysultaline) | | | |
| (50%) | Polymer #1 | Ratio | |
| weight % of | weight % of | surfactant:hydrophobically | Viscosity (cP) RV#6, |
| composition | composition | modified polyurethane | 10-50 rpm, 21° C. |
| 1 | 3 | 0.33 | 16200 |
| 2 | 3 | 0.67 | 36750 |
| 3 | 3 | 1.00 | 37500 |
| 4 | 3 | 1.33 | 29450 |
| 5 | 3 | 1.67 | 14550 |
| 1 | 2 | 0.50 | 2750 |
| 2 | 2 | 1.00 | 4300 |
| 3 | 2 | 1.50 | 1600 |
| 4 | 2 | 2.00 | 200 |
| 5 | 2 | 2.50 | 10 |

### Example 8

### Personal Care Compositions

To test the viscosity of the personal care compositions of the present invention, several exemplary formulations were prepared. Polymer #1 refers to a hydrophobically modified polyurethane that is a copolymer of steareth 100/PEG136/HDI. The viscosities were measured at 21° C. and 10-50 RPM using a Brookfield RV with a #6 spindle.

TABLE 8

| Plantacare | | | |
|---|---|---|---|
| 818 | | | |
| (cocoglucoside) | | | Viscosity |
| (30%) | Polymer #1 | Ratio | (cP) RV#6, |
| weight % of | weight % of | surfactant:hydrophobically | 10-50 rpm, |
| composition | composition | modified polyurethane | 21° C. |
| 1 | 3 | 0.33 | 54100 |
| 2 | 3 | 0.67 | 16550 |
| 3 | 3 | 1.00 | 5250 |
| 4 | 3 | 1.33 | 2750 |
| 5 | 3 | 1.67 | 900 |
| 1 | 2 | 0.50 | 9500 |
| 2 | 2 | 1.00 | 2150 |
| 3 | 2 | 1.50 | 1000 |
| 4 | 2 | 2.00 | 650 |
| 5 | 2 | 2.50 | 450 |

### Example 9

### Personal Care Compositions

To test the effect of the addition of salt on the viscosity of the personal care compositions of the present invention, several exemplary formulations were prepared. Polymer #1 refers to a hydrophobically modified polyurethane that is a copolymer of steareth 100/PEG136/HDI.

TABLE 9A

| | Formulation 1 | Formulation 2 | Formulation 3 | Formulation 4 | Formulation 5 |
|---|---|---|---|---|---|
| Deionized water | 66.00 | 65.00 | 64.25 | 63.00 | 32.00 |
| Disodium laureth sulfsuccinate | 22.00 | 22.00 | 22.00 | 22.00 | 22.00 |
| Betafan-M | 12.00 | 12.00 | 12.00 | 12.00 | 12.00 |
| Polymer #1 | 0 | 1.00 | 1.75 | 2.00 | 3.00 |

Polymer #1 and NaCl were added to base formulation number 2 (described in Table 9A) in the percentages shown in the following Table 9B, resulting in the viscosity shown in the corresponding row in Table 9B.

TABLE 9B

| | Polymer #1 | | |
|---|---|---|---|
| | % addition | NaCl % addition | Viscosity |
| Base #2 | 0 | 0 | 10 |
| Base #2 | 1 | 0 | 15 |
| Base #2 | 1.75 | 0 | 20 |
| Base #2 | 2 | 0 | 30050 |
| Base #2 | 3 | 0 | 74100 |
| Base #2 | 1 | 0 | 15 |
| Base #2 | 1 | 1 | 15 |
| Base #2 | 1 | 2 | 150 |
| Base #2 | 1 | 2.5 | 2500 |
| Base #2 | 1 | 3 | 16500 |
| Base #2 | 1.75 | 0 | 20 |
| Base #2 | 1.75 | 1 | 340 |
| Base #2 | 1.75 | 2 | 27000 |
| Base #2 | 2 | 0 | 30050 |
| Base #2 | 2 | 1 | 160500 |
| Base #2 | 2 | 2 | 219500 |

### Example 10

### Foam Stability of Personal Care Compositions

To test the effect hydrophobically modified polyurethane (polymer #1, a copolymer of steareth 100/PEG136/HDI) on foam stability of personal care compositions of the present invention, hydrophobically modified polyurethane was added to base formulation number 2 (described in Table 9A) in the percentages shown in the following Table 10A, resulting in the foam retention shown in the corresponding row in Table 10A.

Foam stability was determined by blending a 10% solution of surfactant in a waring blender for 1 minute. The solution was transferred to a graduated cylinder and the foam height was recorded. After a period of 3.5 minutes, the foam height was again recorded to indicate stability.

TABLE 10A

| | | | Initial | | | |
|---|---|---|---|---|---|---|
| | Polymer #1 | | volume | After | | % Foam |
| | weight % | Salt | (ml) | 3.5 min. | Drainage | retained |
| Base #2 | 0 | 0 | 250 | 140 | 110 | 44 |
| Base #2 | 2 | 0 | 220 | 145 | 75 | 34 |
| Base #2 | 3 | 0 | 210 | 142 | 68 | 32 |

### Example 11

### Sulfate Free Shampoo

The formulation of a sulfate free shampoo in accordance with the present invention is shown in Table 11A.

TABLE 11A
Shampoo #1 Formulation

| Ingredient | % w/w |
|---|---|
| Deionized water | 55.05 |
| Disodium EDTA (Dissolvine Na2) | 0.10 |
| Gluconolactone | 0.20 |
| Polymer #1 | 1.80 |
| Sodium C14-16 Olefin Sulfonate | 25.00 |
| Cocamidopropyl Betaine | 10.00 |
| Decyl Glucoside | 3.00 |
| Cocoamide MEA | 2.00 |
| Dimethicone PEG-8 Meadowfoamate | 2.00 |
| Methylchloroisothiazolinone (and) | 0.10 |
| Methylisothiazolinone | |
| Ext. Violet 2 | tms |

Combine phase “A” ingredients and heat to 80° C. with propeller mixing. Add phase “B” ingredients to phase “A” one at a time with propeller mixing. Cool to 40° C. with mixing. Add phase “C” to batch. Cool to 30° C. The composition has the appearance of a clear, thick gel with a pH of 5.8-6.2 and a viscosity of 17,000-25,000 cP (measured RV #6 @ 20 RPM).

The effect of hydrophobically modified polyurethane (polymer #1, a copolymer of steareth 100/PEG136/HDI) on foam stability of personal care compositions of the present invention using a sulfate-free shampoo formula in accordance with the present invention as a base formula. Polymer #1 was added to shampoo #1 in the percentages shown in the following Table 11B, resulting in the foam retention shown in the corresponding row in Table 11B.

Foam stability was determined by blending a 10% solution of surfactant/polymer in a waring blender for 1 minute. The solution was transferred to a graduated cylinder and the foam height was recorded. After a period of 3.5 minutes, the foam height was again recorded to indicate stability.

TABLE 11B

| | Initial volume | | | % Foam |
|---|---|---|---|---|
| | (ml) | After 3.5 min. | Drainage | retained |
| Shampoo #1/0% | 390 | 130 | 260 | 67 |
| Polymer #1 | | | | |
| Shampoo #1/ | 370 | 110 | 260 | 70 |
| 1.8% Polymer #1 | | | | |

### Example 12

### Method of Making a Sulfate Free Face Wash

The formulation of a sulfate free face wash in accordance with the present invention is shown in Table 12.

TABLE 12
Face Wash #1 Formulation

| Phase | Ingredient | % w/w |
|---|---|---|
| A | Deionized water | 56.00 |
| | Steareth-100/PEG-136/HDI Copolymer | 2.00 |
| | Hectorite | 1.00 |
| | Coco Glucoside | 14.00 |
| | Sodium Lauroyl Sarcosinate | 2.00 |
| | Meadowfoamamidopropyl Betaine | 14.00 |
| | Glycerin | 2.00 |
| B | PEG-75 Meadowfoam Oil | 1.50 |
| | Meadowfoam Delta-Lactone | 1.00 |
| | Glyceryl Stearate | 2.00 |
| C | Titanium dioxide | 1.00 |
| D | Sodium chloride | 2.00 |
| | Aloe vera | 1.00 |
| | Juglans Regia (walnut) shell powder | a.d. |
| | Phenoxyethanol, ethylhexylglycerin | 0.50 |
| E | Lactic acid | q.s. |
| | Citrus Aurantium Dulcis (orange) Peel | a.d. |
| | Oil (Orange Sweet) | |
| | Citrus Decumana (Grapefruit) Seed Oil | a.d. |

Premix Steareth-100/PEG-136/HDI copolymer in first half of water with moderate speed mixing. Premix Hectorite in second half of water with moderate speed mixing. Combine both batches. Add remaining Phase A ingredients one by one and mix until uniform. Heat Phase A to 75° C. Combine Phase B ingredients and heat to 75° C. Add Phase C to Phase B and mix until uniform. Add Phase B+C to Phase A and mix with propeller until uniform. Cool down to 40° C. with propeller mixing. Add Phase D ingredients one by one. Adjust pH with lactic acid to around 4.5. Add fragrance as desired.

### Example 13

### Method of Making a Sulfate Free Shampoo

The following composition set forth in Table 13 and method are in accordance with an embodiment of the present invention.

TABLE 13

| Phase | Ingredient | Supplier | % w/w range |
|---|---|---|---|
| A | Deionized water | | 40%-60% |
| | Disodium EDTA | Akzo Nobel | 0.01%-0.5% |
| | (dissolving Na2) | | |
| | Gluconolactone | | 0.01%-0.5% |
| | Glycerin | | 0.1%-1.5% |
| | PEG/PPG-450/50 | Elementis | 0.5%-5% |
| | Trimethylolpropane | Specialties | |
| | Dodecyl Ether | | |
| B | Sodium C14-16 Olefin | | 20%-30% |
| | Sulfonate | | |
| | Cocoamidopropyl Betaine | Evonik | 5%-15% |
| | (Tegobetain F50) | | |
| | Decyl Glucoside (Plantacare | BASF | 1%-5% |
| | 2000UP) | | |
| | Cocoamide MEA | | 1%-5% |
| | Dimethicone PEG-8 | Elementis | 1%-5% |
| | MEadowfoamate | Specialties | |
| C | Methylchloroisothiazolinone | Troy Corp. | 0.01%-0.5% |
| | (and) Methylisothiazolinone | | |
| | (Troycare BC04) | | |
| | Ext. Violet 2 | | tms |

Phase “A” ingredients are combined and heated to 80° C. with propeller mixing. Phase “B” ingredients are added to phase “A” one at a time with propeller mixing. The batch is cooled to 40° C. with mixing. Phase “C” is added to batch. The batch is cooled to 30° C. The composition is expected to have the appearance of a clear, thick gel with a pH of 5.8-6.2 and a viscosity of 17,000-25,000 cP.

### Example 14

### Method of Making a Sulfate Free Shampoo

The following composition set forth in Table 14 and method are in accordance with an embodiment of the present invention.

TABLE 14

| Phase | Ingredient | Supplier | % w/w range |
|---|---|---|---|
| A | Deionized water | | 40%-60% |
| | Disodium EDTA | Akzo Nobel | 0.01%-0.5% |
| | (dissolving Na2) | | |
| | Gluconolactone | | 0.01%-0.5% |
| | Glycerin | | 0.1%-1.5% |
| | lauryl - | Elementis | 0.5%-5% |
| | [PEG190/PPO27] × TMP | Specialties | |
| B | Sodium C14-16 Olefin | | 20%-30% |
| | Sulfonate | | |
| | Cocoamidopropyl Betaine | Evonik | 5%-15% |
| | (Tegobetain F50) | | |
| | Decyl Glucoside (Plantacare | BASF | 1%-5% |
| | 2000UP) | | |
| | Cocoamide MEA | | 1%-5% |
| | Dimethicone PEG-8 | Elementis | 1%-5% |
| | MEadowfoamate | Specialties | |
| C | Methylchloroisothiazolinone | Troy Corp. | 0.01%-0.5% |
| | (and) Methylisothiazolinone | | |
| | (Troycare BC04) | | |
| | Ext. Violet 2 | | tms |

Phase “A” ingredients are combined and heated to 80° C. with propeller mixing. Phase “B” ingredients are added to phase “A” one at a time with propeller mixing. The batch is cooled to 40° C. with mixing. Phase “C” is added to batch. The batch is cooled to 30° C. The composition is expected to have the appearance of a clear, thick gel with a pH of 5.8-6.2 and a viscosity of 17,000-25,000 cP.

### Example 15

### Method of Making a Sulfate Free Face Wash

The formulation of a sulfate free face wash in accordance with the present invention is shown in Table 15.

TABLE 15

| Phase | Ingredient | % w/w |
|---|---|---|
| A | Deionized water | 45%-65% |
| | PEG/PPG-450/50 Trimethylolpropane | 0.1%-5% |
| | Dodecyl Ether | |
| | Hectorite | 0.1%-5% |
| | Coco Glucoside | 10%-20% |
| | Sodium Lauroyl Sarcosinate | 0.1%-5% |
| | Meadowfoamamidopropyl Betaine | 10%-20% |
| | Glycerin | 0.1%-5% |
| B | PEG-75 Meadowfoam Oil | 0.1%-5% |
| | Meadowfoam Delta-Lactone | 0.1%-5% |
| | Glyceryl Stearate | 0.1%-5% |
| C | Titanium dioxide | 0.1%-5% |
| D | Sodium chloride | 0.1%-5% |
| | Aloe vera | 0.1%-5% |
| | Juglans Regia (walnut) shell powder | a.d. |
| | Phenoxyethanol, ethylhexylglycerin | 0.1%-5% |
| E | Lactic acid | q.s. |
| | Citrus Aurantium Dulcis (orange) Peel | a.d. |
| | Oil (Orange Sweet) | |
| | Citrus Decumana (Grapefruit) Seed Oil | a.d. |

Premix PEG/PPG-450/50 Trimethylolpropane Dodecyl Ether copolymer in first half of water with moderate speed mixing. Premix hectorite in second half of water with moderate speed mixing. Combine both batches. Add remaining Phase A ingredients one by one and mix until uniform. Heat Phase A to 75° C. Combine Phase B ingredients and heat to 75° C. Add Phase C to Phase B and mix until uniform. Add Phase B+C to Phase A and mix with propeller until uniform. Cool down to 40° C. with propeller mixing. Add Phase D ingredients one by one. Adjust pH with lactic acid to around 4.5. Add fragrance as desired.

### Example 16

### Method of Making a Sulfate Free Face Wash

The formulation of a sulfate free face wash in accordance with the present invention is shown in Table 16.

TABLE 16

| Phase | Ingredient | % w/w |
|---|---|---|
| A | Deionized water | 45%-65% |
| | lauryl - [PEG190/PPO27] × TMP | 0.1%-5% |
| | Hectorite | 0.1%-5% |
| | Coco Glucoside | 10%-20% |
| | Sodium Lauroyl Sarcosinate | 0.1%-5% |
| | Meadowfoamamidopropyl Betaine | 10%-20% |
| | Glycerin | 0.1%-5% |
| B | PEG-75 Meadowfoam Oil | 0.1%-5% |
| | Meadowfoam Delta-Lactone | 0.1%-5% |
| | Glyceryl Stearate | 0.1%-5% |
| C | Titanium dioxide | 0.1%-5% |
| D | Sodium chloride | 0.1%-5% |
| | Aloe vera | 0.1%-5% |
| | Juglans Regia (walnut) shell powder | a.d. |
| | Phenoxyethanol, ethylhexylglycerin | 0.1%-5% |
| E | Lactic acid | q.s. |
| | Citrus Aurantium Dulcis (orange) Peel | a.d. |
| | Oil (Orange Sweet) | |
| | Citrus Decumana (Grapefruit) Seed Oil | a.d. |

Premix lauryl-[PEG190/PP027]xTMP copolymer in first half of water with moderate speed mixing. Premix hectorite in second half of water with moderate speed mixing. Combine both batches. Add remaining Phase A ingredients one by one and mix until uniform. Heat Phase A to 75° C. Combine Phase B ingredients and heat to 75° C. Add Phase C to Phase B and mix until uniform. Add Phase B+C to Phase A and mix with propeller until uniform. Cool down to 40° C. with propeller mixing. Add Phase D ingredients one by one. Adjust pH with lactic acid to around 4.5. Add fragrance as desired.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

I/we claim:

1. A personal care composition comprising:
a surfactant; and
a polymer comprising a hydrophobically modified polyurethane, a hydrophobically modified polyether polyol, or a combination thereof;
wherein the personal care composition has a viscosity ranging from about 8,000 cP to about 40,000 cP; and
wherein the personal care composition is free of sulfates.

2. The personal care composition according to claim 1, wherein the surfactant is non-ionic.

3. The personal care composition according to claim 1, wherein the personal care composition comprises more than one different surfactants.

4. The personal care composition according to claim 3, wherein at least one of the more than one different surfactants is non-ionic.

5. The personal care composition according to claim 1, wherein the at least one surfactant and the polymer are present in a weight ratio of about 0.25:1 to about 3:1.

6. The personal care composition according to claim 1, wherein the polymer is present in an amount ranging from about 1% to about 4% by weight of the personal care composition.

7. The personal care composition according to claim 1, wherein the surfactant is present in an amount ranging from about 0.1% to about 40% by weight of the personal care composition.

8. The personal care composition according to claim 1, wherein the hydrophobically modified polyurethane is selected from condensates of:
(i) at least one polyoxyalkylene with a number average molecular weight ranging from about 2,000-10,000 g/mole;
(ii) a linear, branched, or alicylic aliphatic alcohol having an aliphatic group with 6 to 18 carbon atoms, said aliphatic group optionally linked to a polyoxyalkylene group with a number average molecular weight ranging from about 2,000-10,000 g/mole; and
(iii) an aliphatic diisocyanate compound.

9. The personal care composition according to claim 1, wherein the hydrophobically modified polyurethane is a condensate of a polyethylene glycol with a number average molecular weight of about 4500-6500 g/mole, a linear aliphatic alcohol having an aliphatic group with 18 carbon atoms, said aliphatic group linked to a polyoxyalkylene group with a number average molecular weight of about 3000-5000 g/mole; and hexamethylene diisocyanate.

10. The personal care composition according to claim 8, wherein the condensate further comprises a diol having 6 to 16 carbon atoms.

11. The personal care composition according to any one of claims 8-10, wherein the linear or branched aliphatic alcohol is independently selected from the group consisting of: 1-hexanol, 2-ethylhexanol, 1-octanol, 1-decanol, 1-dodecanol, 1-tetradecanol, 1-hexadecanol, 1-octadecanol, 2-butyloctanol, 2-hexyldecanol, 2-octyldecanol, 1-isononanol, 1-isooctadecanol, 1-isoundecanol, 1-isotridecanol and mixtures thereof.

12. The personal care composition according to claim 11, wherein the diisocyanate compound is independently selected from the group consisting of: hexamethylene diisocyanate, trimethyl hexamethylene diisocyanate, isophorone diisocyanate, tetramethyl xylylene diisocyanate, 4,4′-methylene bis(cyclohexylisocyanate) and mixtures thereof.

13. The personal care composition according to claim 1, wherein the hydrophobically modified polyether polyol is selected from condensates of:
(i) an acyclic polyhydric alcohol having 4 to 6 carbon atoms and 4 to 6 reactive hydroxyl groups; a cyclic polyhydric alcohol having 4 to 6 carbon atoms and 4 to 6 reactive hydroxyl groups, and combinations thereof;
(ii) polyoxyalkylene with a number average molecular weight ranging from about 2,000-10,000 g/mole; and
(iii) a linear, branched, or alicylic aliphatic epoxide having an aliphatic group with 6 to 18 carbon atoms.

14. The personal care composition according to claim 13, wherein the acyclic polyhydric alcohol is selected from pentaerythritol, erythritol, and combinations thereof; and wherein the polyoxyalkylene has alternating groups selected from —(OCH₂CH₂); —(OCH(CH₃)CH₂, —(OCH₂(CH₂)CH₃ and combinations thereof.

15. A method of increasing the viscosity according to a personal care composition comprising:
providing a personal care composition comprising a surfactant and a polymer comprising a hydrophobically modified polyurethane, a hydrophobically modified polyether polyol, or a combination thereof; and
adding a salt to the personal care composition to increase the viscosity of the personal care composition.

16. The method according to claim 15, wherein the at least one surfactant is non-ionic.

17. The method according to claim 15, wherein the hydrophobically modified polyurethane is selected from condensates of:
(i) at least one polyoxyalkylene with a number average molecular weight ranging from about 2,000-10,000 g/mole;
(ii) a linear, branched, or alicylic aliphatic alcohol having an aliphatic group with 6 to 18 carbon atoms, said aliphatic group optionally linked to a polyoxyalkylene group with a number average molecular weight ranging from about 2,000-10,000 g/mole; and
(iii) an aliphatic diisocyanate compound.

18. The method according to claim 17, wherein the hydrophobically modified polyurethane is a condensate of a polyethylene glycol with a number average molecular weight of about 4500-6500 g/mole, a linear aliphatic alcohol having an aliphatic group with 18 carbon atoms, said aliphatic group linked to a polyoxyalkylene group with a number average molecular weight of about 3000-5000 g/mole; and hexamethylene diisocyanate.

19. The method according to claim 17, wherein the condensate further comprises a diol having 6 to 16 carbon atoms.

20. The method according to any one of claims 17-19, wherein the linear or branched aliphatic alcohol is independently selected from the group consisting of: 1-hexanol, 2-ethylhexanol, 1-octanol, 1-decanol, 1-dodecanol, 1-tetradecanol, 1-hexadecanol, 1-octadecanol, 2-butyloctanol, 2-hexyldecanol, 2-octyldecanol, 1-isononanol, 1-isooctadecanol, 1-isoundecanol, 1-isotridecanol and mixtures thereof.

21. The method according to claim 17, wherein the diisocyanate compound is independently selected from the group consisting of: hexamethylene diisocyanate, trimethyl hexamethylene diisocyanate, isophorone diisocyanate, tetramethyl xylylene diisocyanate, 4,4′-methylene bis(cyclohexylisocyanate) and mixtures thereof.

22. The method according to claim 15, wherein the hydrophobically modified polyether polyol is selected from condensates of:
(i) an acyclic polyhydric alcohol having 4 to 6 carbon atoms and 4 to 6 reactive hydroxyl groups; a cyclic polyhydric alcohol having 4 to 6 carbon atoms and 4 to 6 reactive hydroxyl groups, and combinations thereof;
(ii) polyoxyalkylene with a number average molecular weight ranging from about 2,000-10,000 g/mole; and;
(iii) a linear, branched, or alicylic aliphatic epoxide having an aliphatic group with 6 to 18 carbon atoms.

23. The method according to claim 22, wherein the acyclic polyhydric alcohol is selected from pentaerythritol, erythritol, and combinations thereof; and wherein the polyoxyalkylene has alternating groups selected from —(OCH₂CH₂); —(OCH(CH₃)CH₂, —(OCH₂(CH₂)CH₃ and combinations thereof.

24. The method according to claim 15, wherein the salt is added to the composition at about room temperature.

25. The method according to claim 15, wherein the amount of salt added to the composition is sufficient to provide the composition with a viscosity between about 8,000 cP and about 40,000 cP.

26. The method according to claim 15, wherein the salt comprises sodium chloride.

27. The method according to claim 15, wherein the salt is added in an amount of about 0.5 to about 5% by weight of the personal care composition.

28. A method of making a personal care composition comprising:
mixing a polymer comprising a hydrophobically modified polyurethane, a hydrophobically modified polyether polyol, or a combination thereof in water to form a phase A;
heating the phase A to between about 60° C. and about 100° C.;
mixing a surfactant into phase A to form a personal care composition; and
cooling the personal care composition.

29. The method according to claim 28, wherein the at least one surfactant is non-ionic.

30. The method according to claim 28, further comprising mixing an amount of salt to the personal care composition after the cooling step to increase the viscosity of the personal care composition.

31. The method according to claim 30, wherein the amount of salt added to the composition is sufficient to provide the composition with a viscosity between about 8000 cP and about 40000 cP.

32. The method according to claim 30, wherein the salt comprises sodium chloride.

33. The method according to claim 30, wherein the salt is added in an amount of about 0.5 to about 5% by weight of the personal care composition.

34. The method according to claim 28, wherein the hydrophobically modified polyurethane is selected from condensates of:
(i) at least one polyoxyalkylene with a number average molecular weight ranging from about 2,000-10,000 g/mole;
(ii) a linear, branched, or alicylic aliphatic alcohol having an aliphatic group with 6 to 18 carbon atoms, said aliphatic group optionally linked to a polyoxyalkylene group with a number average molecular weight ranging from about 2,000-10,000 g/mole; and
(iii) an aliphatic diisocyanate compound.

35. The method according to claim 34, wherein the hydrophobically modified polyurethane is a condensate of a polyethylene glycol with a number average molecular weight of about 4500-6500 g/mole, a linear aliphatic alcohol having an aliphatic group with 18 carbon atoms, said aliphatic group linked to a polyoxyalkylene group with a number average molecular weight of about 3000-5000 g/mole; and hexamethylene diisocyanate.

36. The method according to claim 34, wherein the condensate further comprises a diol having 6 to 16 carbon atoms.

37. The method according to any one of claims 34-36, wherein the linear or branched aliphatic alcohol is independently selected from the group consisting of: 1-hexanol, 2-ethylhexanol, 1-octanol, 1-decanol, 1-dodecanol, 1-tetradecanol, 1-hexadecanol, 1-octadecanol, 2-butyloctanol, 2-hexyldecanol, 2-octyldecanol, 1-isononanol, 1-isooctadecanol, 1-isoundecanol, 1-isotridecanol and mixtures thereof.

38. The method according to claim 34, wherein the diisocyanate compound is independently selected from the group consisting of: hexamethylene diisocyanate, trimethyl hexamethylene diisocyanate, isophorone diisocyanate, tetramethyl xylylene diisocyanate, 4,4′-methylene bis(cyclohexylisocyanate) and mixtures thereof.

39. The method according to claim 28, wherein the hydrophobically modified polyether polyol is selected from condensates of:
an acyclic polyhydric alcohol having 4 to 6 carbon atoms and 4 to 6 reactive hydroxyl groups; a cyclic polyhydric alcohol having 4 to 6 carbon atoms and 4 to 6 reactive hydroxyl groups, and combinations thereof;
polyoxyalkylene with a number average molecular weight ranging from about 2,000-10,000 g/mole; and;
a linear, branched, or alicylic aliphatic epoxide having an aliphatic group with 6 to 18 carbon atoms.

40. The method according to claim 38, wherein the acyclic polyhydric alcohol is selected from pentaerythritol, erythritol, and combinations thereof; and wherein the polyoxyalkylene has alternating groups selected from —(OCH₂CH₂); —(OCH(CH₃)CH₂, —(OCH₂(CH₂)CH₃ and combinations thereof.
